(19) United States
(12) Patent Application Publication
(10) Pub. No.: US 2010/0216998 A1
(43) Pub. Date: Aug. 26, 2010

(54) Facile "One Pot' Process for Apomorphine From Codeine

(75) Inventor: Narayanasamy Gurusamy, Ballwin, MO (US)

Correspondence Address: Mallinckrodt Inc., 675 McDonnell Boulevard, HAZELWOOD, MO, 63042, US

(73) Assignee: MALLINCKRODT INC., Hazelwood, MO (US)

(21) Appl. No.: 12/678,314

(22) PCT Filed: Sep. 2, 2008
(86) PCT No.: PCT/US2008/075007

§ 371 Date: Mar. 16, 2010

Related U.S. Application Data
(60) Provisional application No. 60/973,251, filed on Sep. 18, 2007.

(51) Int. Cl. C07D 215/14

(52) U.S. Cl. 546/75

(57) ABSTRACT

An improved method for producing apomorphine and derivatives thereof is provided. The method is a convenient ‘one-pot’ process, comprising the conversion of codeine into apomorphine without isolating the apocodeine intermediate. Use of water reactive scavengers, reagents that will react irreversibly with water, decreases side product formation and allows the use of milder reaction conditions. This one-pot synthesis of apomorphine from codeine provides a faster reaction with improved yields at temperatures lower as compared to conventional methods. The lower operating temperatures and less volatile reactants make the method particularly useful for large-scale manufacturing.

### BACKGROUND OF INVENTION

Apomorphine, 5,6,6a,7-tetrahydro-6-methyl-4H-dibenzo[de,g]quinoline-10,11-diol, is a non-narcotic morphine derivative which can be used as pro-emetic agent for accidental poisoning. Apomorphine is also a dopaminergic agonist used to treat “off” episodes in Parkinson Disease patients. Additionally, apomorphine is sold in 46 countries under the trade name Uprima® for the treatment of male erectile dysfunction. More recently, other potential indications for apomorphine, such as female sexual dysfunction, have been disclosed.

Acid-catalyzed morphine/apomorphine type rearrangements are known in the prior art. In conventional synthesis reactions developed prior to 1970, suitable acid catalyst solutions included concentrated HCl, oxalic acid, glacial acetic acid, phosphoric acid, 85% phosphoric acid with flowing anhydrous HCl, 85% phosphoric acid with nitrogen flow through mixture, and concentrated aqueous zinc chloride. Unfortunately, each of these procedures suffered from poor yields, ranging from 0.6% to 46% depending upon the particular acid catalyst and the morphine derivative used. Further, each of these procedures required heating the reaction mixtures to a high temperature, typically about 150° C.

In morphine/apomorphine type rearrangements, one mole of water is eliminated during the course of the rearrangement. In the prior art, the water by-product promoted rearrangements, requiring removal, typically at high temperatures (125° C. to 150° C.) by passing either a current of anhydrous HCl or a current of nitrogen through the reaction mixture. The use of nitrogen led to a cleaner reaction due to oxygen-free atmosphere and avoided the formation of oxidation side-products and chloromorphides that were usually observed when HCl was employed. The yield of apomorphine or apocodeine ranged from 20% to 42%.

U.S. Pat. No. 4,162,361 discloses a method for the preparation of apomorphine or apocodeine in an improved yield, purported to be in the 55% to 70% range. In this method, rearrangement of morphine or codeine takes place in the presence of orthophosphoric acid under a partial vacuum, but requires reaction temperatures from 125° C. to 140° C.

One known method, the methanesulfonic acid catalyzed rearrangement of morphine/apomorphine has been shown to be effective at lower temperatures of about 100° C. However, this reaction is limited in that it only affords yields in the 32% to 35% range.

The common limitations of the processes reported in the prior art for morphine/apomorphine type rearrangement include poor product yield, high reaction temperatures ranging from 125° C. to 150° C., or both. Further, these procedures are cumbersome from the standpoint of unit operations in plant-scale process equipment.

There is therefore a need to develop a more efficient process to carry out this chemistry at an easily achievable reaction temperature. The present invention discloses such a facile, ‘one-pot’ process for making apomorphine and derivatives thereof from codeine.

### SUMMARY OF INVENTION

In an Illustrative aspect of the present invention there is provided a method comprising mixing a compound according to Formula I with at least one acid and at least one water reactive scavenger to form a reaction mixture; heating the reaction mixture to a temperature at which the compound according to Formula I is converted to a compound according to Formula II; and demethylating the compound according to Formula II to form a compound according to Formula III, wherein the compound according to Formula I is converted to the compound according to Formula III in a single reaction vessel without isolating the compound according to Formula II:

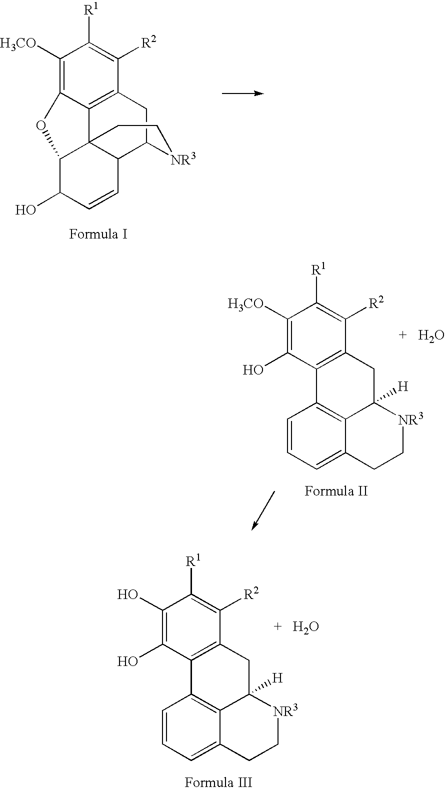

wherein R1, R2 and R3 are independently selected from the group consisting of hydrogen, a halogen, an alkyl substituted alkyl group, a furanyl group, a thienyl group, an alkyl ether, a benzyl group, a substituted benzyl group, a cycloalkyl group and an aryl group.

In another illustrative aspect of the present invention, the compound according to Formula I is codeine, the compound according to Formula II is apocodeine, and the compound according to Formula III is apomorphine.

### DETAILED DESCRIPTION

There is provided an improved and convenient ‘one-pot’ process for the synthesis of apomorphine, and derivatives thereof, consisting of carrying out the rearrangement of codeine into apocodeine and subsequent demethylation of apocodeine into apomorphine within one reaction vessel. Recent experimentation has unexpectedly determined that the one-pot synthesis of apomorphine from codeine provides a faster reaction with improved yields even at temperatures lower than utilized in conventional methods. The lower temperatures as well as the use of less volatile reactants are greatly preferred for manufacturing. Heretofore, conventional knowledge has held that converting morphine to apomorphine would be a simpler, more efficient synthesis route.

The general reaction scheme of the present invention is given below:

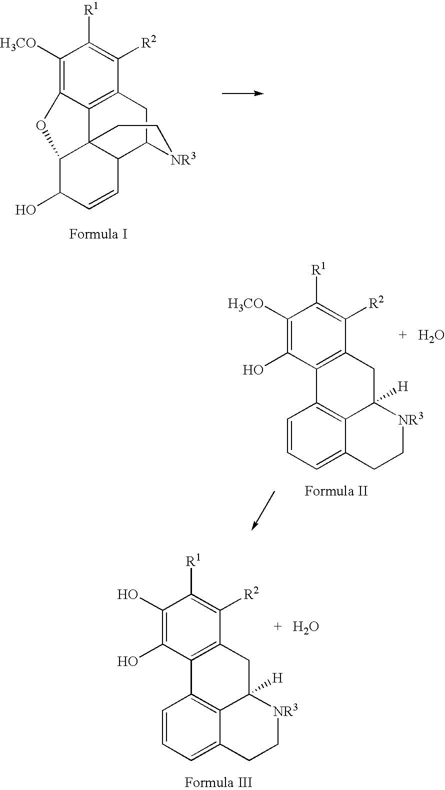

wherein R1, R2 and R3 are independently selected from the group consisting of hydrogen, a halogen, an alkyl substituted alkyl group, a furanyl group, a thienyl group, an alkyl ether, a benzyl group, a substituted benzyl group, a cycloalkyl group and an aryl group.

A compound of Formula I is mixed with an acid and a water reactive scavenger to form a reaction mixture. The reaction mixture is then heated to a temperature at which a substantial portion of the compound of Formula I is converted to the compound of Formula II. As the conversion progresses, the water produced is bound by the scavenger, resulting in fewer rearrangement by-products, and allowing the use of lower reaction temperatures.

In one embodiment, the reaction mixture is heated to less than or about 110° C. In another embodiment the reaction mixture is heated to a temperature from about 65° C. to about 110° C. In still another embodiment, the reaction mixture is heated to a temperature from about 85° C. to about 110° C. The compound of Formula II is then demethylated to form the compound of Formula III. Formula I is converted into Formula Ill in a single vessel without isolation of Formula II, while maintaining high product yield. In another illustrative embodiment, the reaction mixture is maintained under an inert atmosphere typically nitrogen, to prevent the formation of oxidative side products.

Suitable acids for use in the present invention include any acid that will promote the dehydrative rearrangement of morphine type alkaloids, as are known in the art. Illustrative examples include but are not limited to phosphoric acid, methanesulfonic acid and mixtures thereof.

Suitable water reactive scavengers include reagents that will react irreversibly with water under the instant reaction conditions. Illustrative examples include but are not limited to phosphorus pentoxide, polyphosphoric acids, anhydrides such as phthalic anhydride, orthoesters, hexamethyldisilazane, titanium chloride, and mixtures thereof. In one embodiment, the water reactive scavenger is an anhydride.

Conventional demethylation reagents may be utilized for the demethylation step. Illustrative examples include but are not limited to boron tribromide, aluminum chloride, methionine in methanesulfonic acid, pyridine hydrochloride and mixtures thereof. Alternatively, dilute HCl may be employed for demethylation. An array of halide salts such as bromides and iodides may optionally be added to promote the efficiency of O-demethylation in dilute HCl medium. Illustrative examples include but are not limited to LiBr, NaBr, MgBr2 and phase transfer halides such as tetrabutylammonium bromide, as well as mixtures thereof.

In an illustrative example, apomorphine is prepared by heating codeine in methanesulfonic acid in the presence of a suitable water reactive scavenger such as acetic anhydride. The use of a calculated amount of acetic anhydride, enough to consume the water produced in the reaction, obviates the need for temperatures above about 110° C., and improves yields into the at least about 55% range, with yields as high as over 90% observed. The amount of acetic anhydride required is based on the fact that codeine contains one mole of water as water of hydration, and apomorphine transformation generates another mole of water, Therefore two moles of acetic anhydride per mole of codeine are required.

### EXAMPLES

### Example 1

A mixture of 100.00 g codeine monohydrate, 500.00 g methanesulfonic acid and 35.00 g acetic anhydride was heated at 90-95° C. under an inert atmosphere. The resulting solution was stirred at 90-95° C. for 30 minutes to form apocodeine. The reaction mixture containing apocodeine was heated with 48% aqueous HBr at 110-112° C. to obtain apomorphine of 93% analytical yield. Pure apomorphine hydrochloride salt was isolated by subjecting the reaction mixture to salting out, pH adjusting to liberate free base, extraction of free base and conversion into hydrochloride salt. MS date: [M+H]=268. H1 and C14 NMR date substantiated the structural assignment of apomorphine and these spectra perfectly matched the reference spectra recorded in Aldrich Library of NMR.

### Example 2

A mixture of 5.0 g codeine monohydrate, 22.2 g methanesulfonic acid and 7.0 g of Eaton's Reagent (7.7 weight % phosphorus pentoxide in methanesulfonic acid) was heated at 90° C. under an inert atmosphere. The resulting solution was stirred at 90° C. to form apocodeine of 69% analytical yield. The reaction mixture containing apocodeine was heated with 48% aqueous HBr at 100-105° C. to obtain apomorphine of 61% analytical yield.

Unless otherwise noted, all percentages herein are weight percentages.

Having described the invention in detail, those skilled in the art will appreciate that modifications may be made of the invention without departing from its' spirit and scope. Therefore, it is not intended that the scope of the invention be limited to the specific embodiments described. Rather, it is intended that the appended claims and their equivalents determine the scope of the invention.

What is claimed is:

1. A method comprising:
a) mixing a compound according to Formula I with at least one acid and at least one water reactive scavenger to form a reaction mixture; and
b) heating the reaction mixture to a temperature at which the compound according to Formula I is converted to a compound according to Formula II; and
c) demethylating the compound according to Formula II to form a compound according to Formula III, wherein the compound according to Formula I is converted to a compound according to Formula III in a single reaction vessel without isolating the compound according to Formula II; 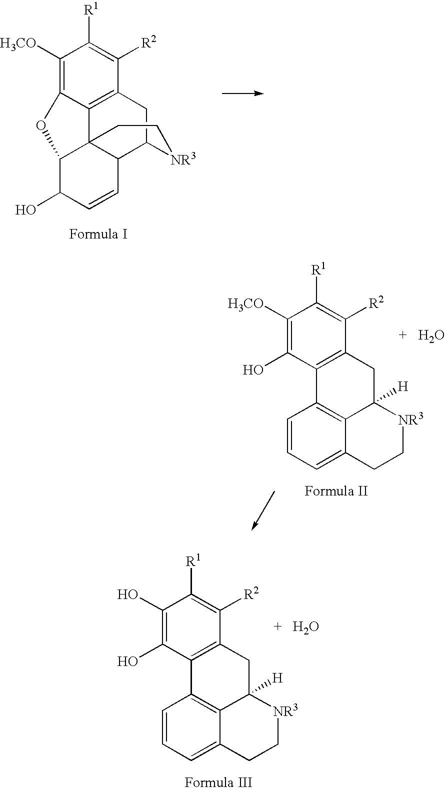
wherein R1, R2 and R3 are independently selected from the group consisting of hydrogen, a halogen, an alkyl substituted alkyl group, a furanyl group, a thienyl group, an alkyl ether, a benzyl group, a substituted benzyl group, a cycloalkyl group and an aryl group.

2. The method according to claim 1 wherein the acid is selected from the group consisting of phosphoric acid, methanesulfonic acid and mixtures thereof.

3. The method according to claim 1 wherein the water reactive scavenger is selected from the group consisting of phosphoric pentoxide, polysphosphoric acids, anhydrides, orthoesters, hexamethyldisilazane and titanium chloride.

4. The method according to claim 1 wherein the compound according to Formula I is codeine; the compound according to Formula II is apocodeine and the compound according to Formula III is apomorphine.

5. The method according to claim 1 wherein the temperature is less than or about 110° C.

6. The method according to claim 1 further including maintaining the reaction mixture in an inert atmosphere.

7. The method according to claim 1 wherein the acid is methanesulfonic acid; the water reactive scavenger is acetic anhydride; and the temperature is less than or about 110° C.

8. The method according to claim 1 wherein the yield of the compound according to Formula III is at least about 55%.

9. A dosage form including a compound according to Formula III, wherein the compound according to Formula III is produced by the method of claim 1.

10. A method for making apomorphine, the method comprising
a) mixing codeine with at least one acid and at least one water reactive scavenger to form a reaction mixture;
b) heating the reaction mixture to a temperature at which the codeine is converted to apocodeine; and
c) demethylating the apocodeine to form apomorphine; wherein the codeine is converted into apomorphine in a single reaction vessel without isolation of the apocodeine.

11. The method according to claim 10 wherein the acid is methanesulfonic acid; the water reactive scavenger is acetic anhydride; and the temperature is less than or about 110° C.

12. The method according the claim 10 further including maintaining the reaction mixture under an inert atmosphere.

13. The method according to claim 10 wherein the codeine is converted to apomorphine with a yield of at least about 55%.

14. A dosage form comprising apomorphine produced by the method of claim 10.
